(19) United States
(12) Patent Application Publication
(10) Pub. No.: US 2026/0272851 A1
(43) Pub. Date: Sep. 17, 2026

(54) FORMULATION COMPRISING APOCYNIN AND PAEONOL

(71) Applicant: AKL Research & Development Ltd, Manchester (GB)

(72) Inventors: Nicholas John LARKINS, London (GB); Alan REYNOLDS, Eastleigh (GB)

(21) Appl. No.: 19/134,275

(22) PCT Filed: Dec. 1, 2023
(86) PCT No.: PCT/GB2023/053119

§ 371 Date: May 30, 2025

(30) Foreign Application Priority Data
Dec. 2, 2022 (GB) 2218160.6

(51) Int. Cl. A61K 31/12

(52) U.S. Cl. CPC A61K 31/12

(57) ABSTRACT

The present invention relates to a composition comprising 2-hydroxy-4-methoxyacetophenone (paconol) or an isomer thereof and 4-hydroxy-3-methoxyacetophenone (apocynin) or an isomer thereof for use in the treatment of senescence e.g. by a senomorphic mechanism of action.

The present invention relates to a composition comprising 2-hydroxy-4-methoxyacetophenone (paeonol) or an isomer thereof and 4-hydroxy-3-methoxyacetophenone (apocynin) or an isomer thereof for use in the treatment of senescence e.g. by a senomorphic mechanism of action.

Cellular senescence is important for the maintenance of tissue homeostasis. Senescence, as a general term, refers to biological ageing. Dysregulation of senescence is linked to many human diseases as well as the ageing process which is the major risk factor for debilitating conditions including cancer, cardiovascular disease and neurodegenerative diseases.

Cellular senescence is a mechanism that permanently arrests the growth of cells. Senescence can be provoked by stresses such as telomere erosion resulting from repeated cell division, mitochondrial deterioration, DNA damage and oxidative stress.

Because cellular senescence is characterised by the cessation of cell division, it ensures that aged or damaged cells do not perpetuate their genomes. This can prevent carcinogenesis in normal development, however, senescent cells accumulate and produce proinflammatory and inflammatory cytokines, chemokines and enzymes, together known as senescence-associated secretory phenotype (SASP) factors. SASP factors are thought to disrupt tissue structure and function, and play a significant role in the development of age-related diseases. These include, but are not limited to, osteoporosis; sarcopenia; lung diseases such as chronic obstructive pulmonary disease (COPD), idiopathic pulmonary fibrosis (IPF), pulmonary hypertension (PH) and pulmonary arterial hypertension (PAH); liver diseases such as acute liver injury, chronic liver disease and hepatic steatosis; kidney diseases such as acute kidney injury (AKI), chronic kidney disease (CKD), tubulointerstitial fibrosis (TIF), glomerulosclerosis and vascular sclerosis; vascular diseases such as atherosclerosis; type 1 diabetes; age-related macular degeneration; and neurodegenerative diseases such as Alzheimer's disease (AD), Parkinson's disease, frontotemporal dementia, amyotrophic lateral sclerosis and multiple sclerosis.

Osteoporosis is an age-related skeletal disorder characterised by low bone mass and progressive deterioration of bone tissue, leading to increased fracture risk. It has been linked to the accumulation of senescent cells in bone (osteocytes). The overproduction of proinflammatory cytokines by senescent osteocytes contributes to chronic inflammation and creates a toxic microenvironment that causes age-related bone loss. The elimination of senescent cells and/or the SASP holds promise for slowing or preventing age-related osteoporosis.

Sarcopenia is an age-related musculoskeletal disorder involving the progressive loss of muscle mass, strength and function. Senescence of the muscle stem cells is thought to cause loss of muscle regenerative potential, while increased levels of SASP factors secreted by senescent cells cause chronic inflammation, which may accelerate protein degradation and muscle fibre loss, resulting in sarcopenia. Elimination of senescent cells and/or the SASP has potential for the treatment of sarcopenia.

Several lung diseases have been linked to senescence; in particular, chronic obstructive pulmonary disease (COPD), an inflammatory lung disease causing progressive limitation of airflow to the lungs; idiopathic pulmonary fibrosis (IPF), a chronic and fatal lung disease in which the tissue surrounding the alveoli in the lungs becomes scarred; pulmonary hypertension (PH) and pulmonary arterial hypertension (PAH). The accumulation of senescent fibroblasts and lung epithelial cells, and the SASP, have been strongly linked to lung disease pathology. Elimination of senescent cells and/or the SASP may have potential as a therapeutic strategy to treat COPD, IPF, PH and PAH.

Liver conditions have also been linked to senescence, particularly acute liver injury, chronic liver disease and hepatic steatosis (the accumulation of fat in the liver that is unrelated to alcohol consumption). A close correlation has been found between markers of senescence in hepatocytes and acute liver injury, chronic liver disease, hepatic fat accumulation and steatosis. Therefore, elimination of senescent cells and/or the SASP may have potential to treat liver conditions.

Kidney diseases, particularly acute kidney injury (AKI), chronic kidney disease (CKD), tubulointerstitial fibrosis (TIF), glomerulosclerosis and vascular sclerosis, have been strongly linked to cellular senescence. The elimination of senescent cells and/or the SASP could be a new therapeutic approach for kidney diseases.

Atherosclerosis is a chronic disease of the arteries characterised by the excessive deposition of fatty material (plaques, or atheroma) on the inner arterial walls. Senescence of lipid-loaded macrophages (‘foam cells’) is shown to be detrimental at all stages of atherosclerosis. The accumulation of senescent foam cells in the subendothelial space at the onset of atherosclerosis is thought to promote atheroma formation and maturation through the SASP. Therefore, elimination of senescent cells and/or the SASP may have potential for the treatment of atherosclerosis.

Type 1 diabetes (T1D) is an autoimmune disease characterised by the progressive loss of pancreatic beta cells, resulting in hyperglycemia. The pathogenesis of T1D has been strongly linked to the senescence of beta cells. Targeted elimination of senescent beta cells has been shown to prevent T1D, and could be a viable approach for T1D treatment.

Age-related macular degeneration (AMD) is an age-related condition of the macula area of the eye that affects central vision. Senescence in retinal and choroidal tissue cells is shown to be a significant contributor to the onset and development of AMD. Elimination of senescent cells and/or the SASP may have the potential to treat AMD.

Senescence in astrocytes, microglia, oligodendrocytes, neurons and neural stem cells are involved in the development of neurodegenerative diseases including Alzheimer's disease, Parkinson's disease, frontotemporal dementia, amyotrophic lateral sclerosis and multiple sclerosis.

Diverse studies in mice and humans suggest that targeting senescent cells and/or the SASP could be a powerful strategy to delaying age-related pathologies, improving healthspan and potentially improving lifespan.

Senescence can be targeted by senotherapeutic compounds, which are divided into two classes: senolytic and senomorphic compounds. A compound is said to be senolytic when its administration results in the selective elimination of senescent cells, and therefore a decrease in both the number of senescent cells and the total number of cells. A senomorphic compound is a compound whose administration results in the selective suppression of the SASP, and a maintenance or increase in the total number of cells.

There is a need for a composition to be used in the treatment of senescence, given the pivotal role of senescent cells and the SASP as risk factors for age-related pathologies.

According to the present invention there is provided a composition comprising 2-hydroxy-4-methoxyacetophenone (paeonol) or an isomer thereof and 4-hydroxy-3-methoxyacetophenone (apocynin) or an isomer thereof for use in the treatment of senescence, wherein administration of the composition results in a decrease in the number of senescent cells and a simultaneous increase in the total viable cell number. Herein the term ‘viable’ means cells that are living and capable of reproduction. Cell viability may be assessed by means of staining with viability dyes such as trypan blue or Calcein-AM, or staining with Hoechst stain followed by the counting of intact nuclei. The composition according to the invention may produce its effect by a senolytic mechanism of action or a senomorphic mechanism of action. Preferably the composition according to the invention produces its effect by a senomorphic mechanism of action. Preferably the composition according to the invention produces its effect by a senomorphic mechanism of action which results in the selective suppression of the SASP.

According to the present invention in a further aspect there is provided a composition comprising paeonol or an isomer thereof and apocynin or an isomer thereof for use in the treatment of senescence by a senomorphic mechanism of action.

According to the present invention in a further aspect there is provided a composition comprising paeonol or an isomer thereof and apocynin or an isomer thereof for use in the treatment of senescence by a senolytic mechanism of action.

Paeonol is 2-hydroxy-4-methoxyacetophenone and is shown by the following formula:

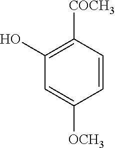 [SMILES: COc1ccc(C(C)=O)c(O)c1]

Paeonol may be found in plant substances and plant extracts. For example paeonol may be found in Paeonia suffruticosa Paeonia lactiflora, Paeonia veitchii, Paeonia obovata, Rheum palmatum (rhizome) and Scutellaria baicalensis (root). The compositions (and medicaments) of the invention include 2-hydroxy-4-methoxy-acetophenone (paeonol).

Paeonol is a solid with a low melting point of around 49.7° C. according to our studies.

Preferably, paeonol has been synthesised or extracted from plants and purified. This may be referred to as isolated paeonol. The quantities and ratios described herein refer to isolated paeonol. Less preferably, paeonol may be present in compositions (and medicaments) according to the invention as direct extracts from plants (i.e. as part of an unresolved mixture of compounds in the form of an unpurified plant or root extract). These will be referred to a paeonol “in the natural form” or “natural paeonol”. For example, paeonol present in compositions (and medicaments) according to the invention in the form of Paeonia suffruticosa will be referred to as “natural paeonol”. The terms paeonol “in the natural form” or “natural paeonol” include glycosides of paeonol such as those found in the plant species in which paeonol is found. Such glycosides include paeonin, paeonolide and paeonoside, for example. If natural paeonol is used the skilled person would readily understand how to adjust the ratios to provide a formulation of the invention.

Isolated paeonol provides a more consistent quality of paeonol compared to natural paeonol. It can also be produced on a larger scale to that of natural paeonol. Consequently, isolated paeonol is preferably used in the present application.

Apocynin is a plant phenol 4-hydroxy-3-methoxyacetophenone, and has the following formula:

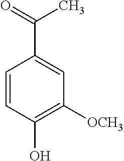

Apocynin is a solid with a much higher melting point of around 114.6° C. according to our studies.

Apocynin is found in plant substances and plant extracts, for example in extracts of the plants Picrorrhiza kurroa, Apocynum cannabinium, Apocynum venatum, Apocynum androsaemifolium and vanilla species such as Vanilla planifolia.

The compositions (and medicaments) of the invention include 4-hydroxy-3-methoxyacetophenone (apocynin). Preferably the apocynin has been synthesised, or extracted from plants and purified. This may be referred to as isolated apocynin. The quantities and ratios described herein refer to isolated apocynin.

Apocynin may be present in compositions (and medicaments) according to the invention as direct extracts from plants such as those mentioned above (for example as part of an unresolved mixture of compounds in the form of an unpurified plant or root extract). These will be referred to as apocynin “in the natural form” or “natural apocynin”. For example, apocynin present in compositions (and medicaments) according to the invention in the form of Picrorrhiza kurroa will be referred to as “natural apocynin”. The term “natural apocynin” or apocynin “in the natural form” also includes glycosides of apocynin such as those found in the plant species in which apocynin is found. Such glycosides include androsin and other iridoid glycosides, for example.

The composition may include apocynin as part of an unresolved mixture of compounds in the form of an unpurified plant or root extract: “natural” apocynin. Picrorrhiza kurroa is a standardised form based on standardised iridoid glucoside fraction; such forms are well known.

A Picrorrhiza kurroa in standardised form comprises Picrorrhiza kurroa standardised to “Kutkin min 4%”. Kutkin is obtained by crystallization and consists of the glucosides picroside I and kutoside in a ratio of 1:2 and other minor glycoside (Sing and Rastogi, 1972, Ansari et al., 1988).

Isolated apocynin provides a more consistent quality of apocynin compared to natural apocynin. It can also be produced on a larger scale to that of natural apocynin. Consequently, isolated apocynin is preferably used in the present invention.

As indicated above, the composition may include apocynin which is in the natural form, although this is less preferred. If natural apocynin is used the skilled person would readily understand how to adjust the ratios to produce a formulation of the invention. However, if this is the case it may be necessary to limit the amount of Picrorrhiza kurroa to prevent side effects (such as stomach upset which may occur due to other phytochemical species in the Picrorrhiza kurroa). However, it is noted that most human subjects can take up to 2,000 mg of Picrorrhiza kurroa (Kutkin min 2%) per day without discomfort.

Isomers of paeonol and apocynin are known in the art. Isomers of paeonol and apocynin include 2-hydroxy-3-methoxyacetophenone “orthoacetovanillone” (CAS: 703-98-0), 2-hydroxy-5-methoxyacetophenone (CAS: 705-15-7), 3-hydroxy-4-methoxyacetophenone “isoacetovanillone” (CAS: 6100-74-9) and 4-hydroxy-2-methoxyacetophenone “isopaeonol” (CAS: 493-33-4).

Paeonol and apocynin are isomers of each other. It will be appreciated that according to the present invention the composition must comprise two different active compounds (e.g. paeonol itself and apocynin itself). Preferably the composition according to the invention comprises paeonol itself and apocynin itself. Herein, the name “APPA” is used to denote a composition comprising paeonol and apocynin, as indicated above.

The applicants have found that in human chondrocyte cell lines, the compositions of the invention e.g. APPA had the capacity to decrease the number of senescent cells.

The applicants have further found that administration of the compositions of the invention e.g. APPA to human chondrocyte cell lines caused an increase in total cell number. The compositions of the invention e.g. APPA therefore exhibit a senomorphic effect on human chondrocyte cell lines (see the Examples).

The use of a paeonol or an isomer thereof with apocynin or an isomer thereof as a senolytic and/or senomorphic medicament can be seen as a remarkable advance allowing for new therapeutic avenues in the elimination or delaying of the adverse effects of cellular senescence, and consequently age-associated pathologies.

The composition according to the invention may have a ratio (by weight) of paeonol or an isomer thereof to apocynin or an isomer thereof from 3:2 to 9:1. For example, the composition according to the invention may have a ratio (by weight) of paeonol or an isomer thereof to apocynin or an isomer thereof from 3:2 to 5:1. For example, the composition according to the invention may have a ratio (by weight) of paeonol or an isomer thereof to apocynin or an isomer thereof from 5:1 to 9:1.

The composition according to the invention may be a liquid formulation. The composition according to the invention may be a liquid formulation made from a eutectic mixture comprising active compounds paeonol or isomer thereof and apocynin or isomer thereof, the liquid formulation further comprising at least one excipient. The excipient may be a glycol or glycol derivative, a polyhydric alcohol, or an ester and/or ether thereof. In one embodiment the formulation has a ratio by weight of total active compounds to excipient from 2:3 to 19:1. Preferably, the ratio by weight of total active compounds to excipient is from 2:3 to 9:1. Preferably, the ratio by weight of total active compounds to excipient is 64:36. Preferably the formulation is a stable liquid at room temperature (15 to 25° C.).

The composition according to the invention may be a solid formulation, such as a powder, tablet or pill.

The composition according to the invention may be used to treat age-related diseases.

The composition according to the invention may be used to treat osteoporosis. The composition according to the invention may be used to treat sarcopenia. The composition according to the invention may be used to treat chronic obstructive pulmonary disease (COPD). The composition according to the invention may be used to treat idiopathic pulmonary fibrosis (IPF).

The composition according to the invention may be used to treat lung diseases such as pulmonary hypertension (PH) and pulmonary arterial hypertension (PAH). The composition according to the invention may be used to treat liver diseases such as acute liver injury, chronic liver disease and hepatic steatosis. The composition according to the invention may be used to treat kidney diseases such as acute kidney injury (AKI), chronic kidney disease (CKD), tubulointerstitial fibrosis (TIF), glomerulosclerosis, and vascular sclerosis. The composition according to the invention may be used to treat vascular diseases such as atherosclerosis. The composition according to the invention may be used to treat type 1 diabetes. The composition according to the invention may be used to treat age-related macular degeneration (AMD). The composition according to the invention may be used to treat Alzheimer's disease (AD), Parkinson's disease, frontotemporal dementia, amyotrophic lateral sclerosis or multiple sclerosis.

The composition according to the invention may be administered orally.

The pharmaceutical compositions of the present invention can be formulated into well-known compositions for any route of drug administration, e.g. oral, rectal, parenteral, transdermal (e.g. patch technology or a transdermal gel formulation), intravenous, intramuscular, subcutaneous, intracisternal, intravaginal, intraperitoneal, local (powders, ointments or drops), as a buccal or nasal spray. A typical composition comprises a pharmaceutically acceptable carrier, such as aqueous solution, non-toxic excipients, including salts and preservatives, buffers and the like, as described in Remington's Pharmaceutical Sciences fifteenth edition (Matt Publishing Company, 1975), at pages 1405 to 1412 and 1461-87, and the national formulary XIV fourteenth edition (American Pharmaceutical Association, 1975), among others.

Examples of suitable aqueous and non-aqueous pharmaceutical carriers, diluents, solvents or vehicles include water, ethanol, polyols (such as glycerol, propylene glycol, polyethylene glycol, and the like), carboxymethylcellulose and suitable mixtures thereof, vegetable oils (such as olive oil), and injectible organic esters such as ethyl oleate.

The compositions of the present invention also can contain additives such as but not limited to preservatives, wetting agents, emulsifying agents, and dispersing agents. Antibacterial and antifungal agents can be included to prevent growth of microbes and includes, for example, paraben, chlorobutanol, phenol, sorbic acid, and the like. Furthermore, it may be desirable to include isotonic agents such as sugars, sodium chloride, and the like.

According to the present invention in a further aspect there is provided the use of apocynin or an isomer thereof and paeonol or an isomer thereof in the manufacture of a medicament for the treatment of senescence.

According to the present invention in a further aspect there is provided the use of apocynin or an isomer thereof and paeonol or an isomer thereof in the manufacture of a medicament for the treatment of age-related diseases.

According to the present invention in a further aspect there is provided the use of apocynin or an isomer thereof and paeonol or an isomer thereof in the manufacture of a medicament for the treatment of osteoporosis; sarcopenia; lung diseases such as chronic obstructive pulmonary disease (COPD), idiopathic pulmonary fibrosis (IPF), pulmonary hypertension (PH) and pulmonary arterial hypertension (PAH); liver diseases such as acute liver injury, chronic liver disease and hepatic steatosis; kidney diseases such as acute kidney injury (AKI), chronic kidney disease (CKD), tubulointerstitial fibrosis (TIF), glomerulosclerosis and vascular sclerosis; vascular diseases such as atherosclerosis; type 1 diabetes; age-related macular degeneration (AMD); and neurodegenerative diseases such as Alzheimer's disease (AD), Parkinson's disease, frontotemporal dementia, amyotrophic lateral sclerosis and multiple sclerosis.

According to the present invention in a further aspect there is provided the use of apocynin or an isomer thereof and paeonol or an isomer thereof in the manufacture of a medicament for the treatment of Alzheimer's disease, Parkinson's disease, frontotemporal dementia, amyotrophic lateral sclerosis or multiple sclerosis.

According to the present invention there is provided a method of treatment of senescence, the method comprising a step of administering to a patient in need thereof a composition comprising apocynin or an isomer thereof and paeonol or an isomer thereof.

According to the present invention there is provided a method of treatment of age-related diseases, the method comprising a step of administering to a patient in need thereof a composition comprising apocynin or an isomer thereof and paeonol or an isomer thereof.

According to the present invention in a further aspect there is provided a method of treatment of osteoporosis, sarcopenia, lung diseases such as chronic obstructive pulmonary disease (COPD), idiopathic pulmonary fibrosis (IPF), pulmonary hypertension (PH) and pulmonary arterial hypertension (PAH), liver diseases such as acute liver injury, chronic liver disease and hepatic steatosis, kidney diseases such as acute kidney injury (AKI), chronic kidney disease (CKD), tubulointerstitial fibrosis (TIF), glomerulosclerosis and vascular sclerosis, vascular diseases such as atherosclerosis, type 1 diabetes, age-related macular degeneration (AMD), Alzheimer's disease (AD), Parkinson's disease, frontotemporal dementia, amyotrophic lateral sclerosis or multiple sclerosis; the method comprising a step of administering to a patient in need thereof a composition comprising apocynin or an isomer thereof and paeonol or an isomer thereof.

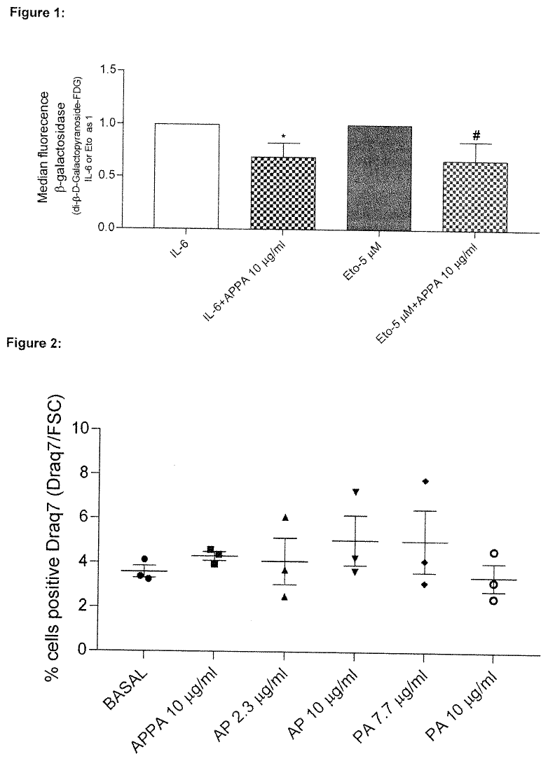

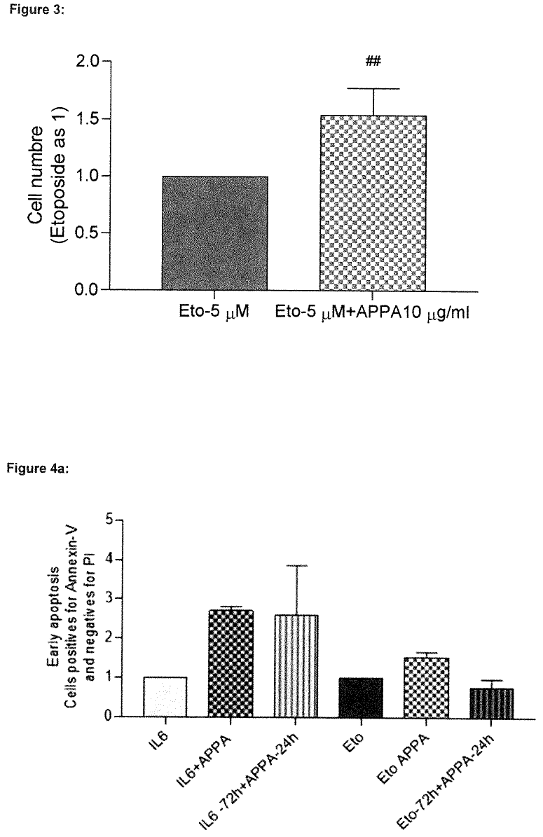

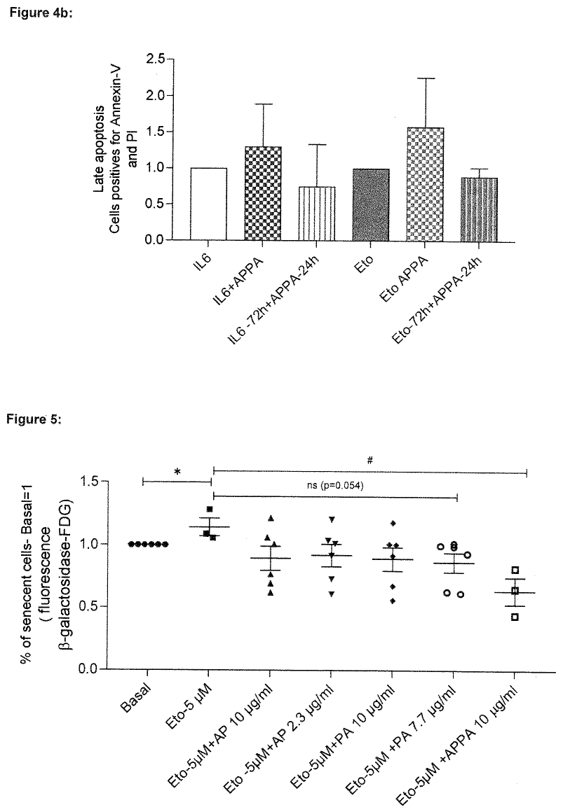

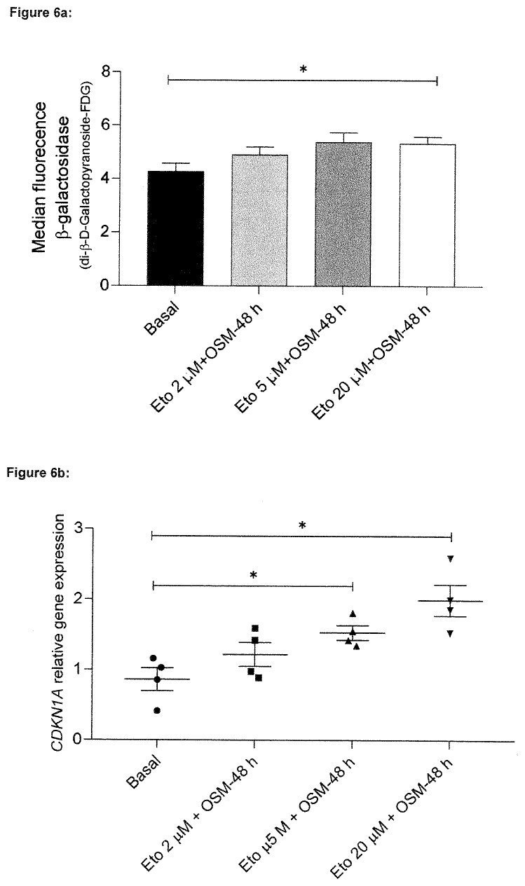

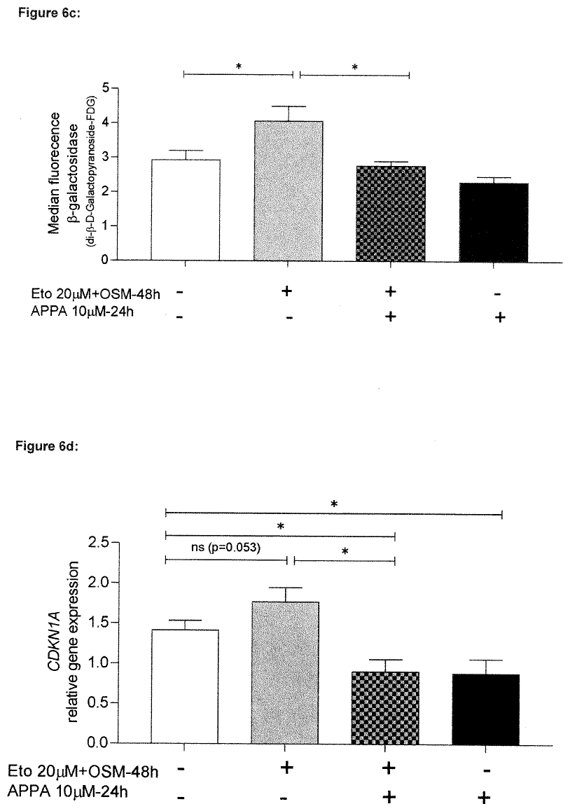

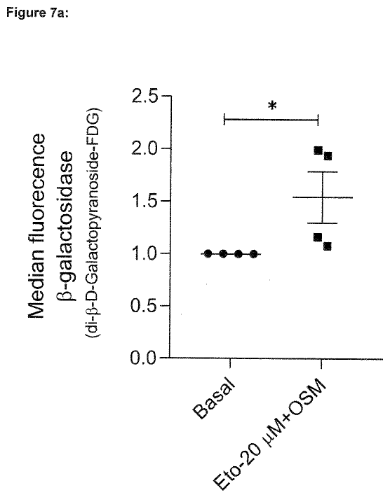

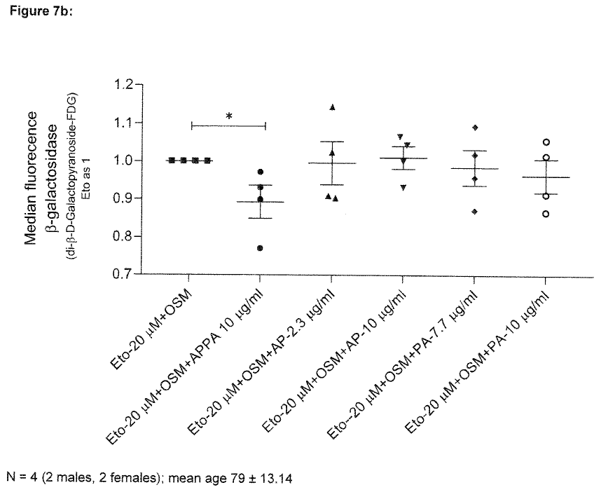

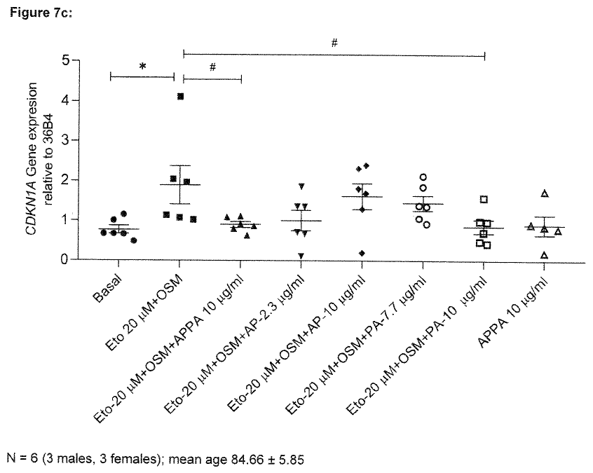

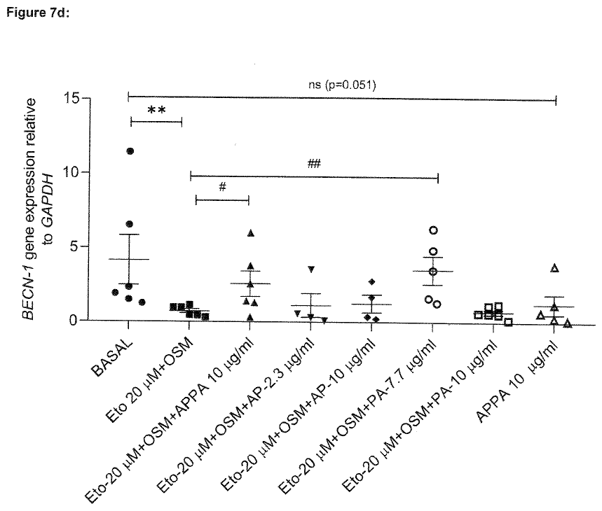

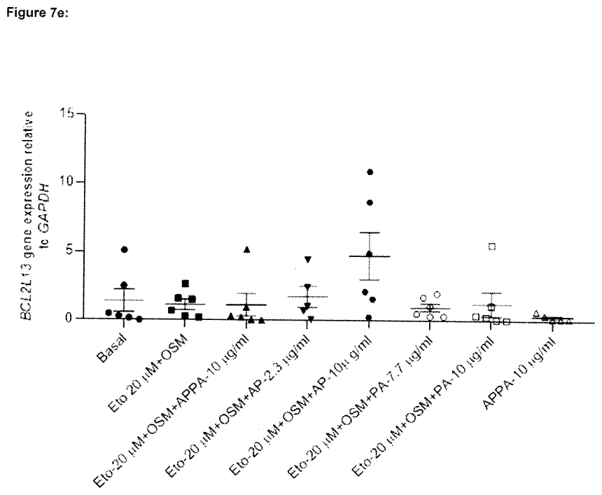

The present invention will now be described in detail with reference to the following examples and to the attached drawings in which:
Example 1 demonstrates the senomorphic activity of APPA in chondrocyte cell line TC28a2;
Throughout the Examples, ‘*’ means ‘in comparison to Basal measurement’; ‘#’ means ‘in comparison to etoposide only measurement’;
FIG. 1 shows the effect of APPA on the levels of the Senescent Associated Secretory Phenotype (SASP) marker β-Galactosidase in TC28a2 chondrocyte cells when induced by exposure to IL-6 or Etoposide (Eto);
FIG. 2 shows the effect of APPA, AP and PA on chondrocyte viability using DRAQ7, in human TC28a2 chondrocyte cells;
FIG. 3 shows the effect of APPA the number of viable TC28a2 cells exposed to Etoposide (Eto);
FIG. 4 shows the effects of APPA and early and late TC28a2 apoptosis in TC28a2 cells exposed to IL-6 or Etoposide (Eto);
FIG. 4a: Effects of APPA, AP and PA on early apoptosis in human TC28a2 chondrocyte cells;
FIG. 4b: Effects of APPA, AP and PA on late apoptosis in human TC28a2 chondrocyte cells;
FIG. 5 shows the effect of APPA and its components, AP and PA, on the levels of the Senescent Associated Secretory Phenotype (SASP) marker β-Galactosidase in TC28a2 chondrocyte cells when induced by exposure to Etoposide;
Example 2 demonstrates the senomorphic activity of APPA in primary human chondrocytes;
FIG. 6 shows the effects of APPA on SA-β-Gal activity and CDKN1A gene expression levels in primary human chondrocytes exposed to etoposide and OSM;
FIG. 6a: Effects of varying concentrations of etoposide (+OSM) on levels of the Senescent Associated Secretory Phenotype (SASP) marker β-Galactosidase;
FIG. 6b: Effects of varying concentrations of etoposide (+OSM) on CDKN1A gene expression in primary human chondrocytes;
FIG. 6c: Effects of APPA on the increased levels of the Senescent Associated Secretory Phenotype (SASP) marker β-Galactosidase induced by 20 μM Etoposide (Eto) plus 10 ng/ml Oncostatin M (OSM); ‘+’ means ‘present’; ‘-’ means ‘absent’
FIG. 6d: effects of APPA on the increased relative gene expression of CDKN1A induced by 20 μM Etoposide (Eto) plus 10 ng/ml Oncostatin M (OSM); ‘+’ means ‘present’; ‘-’ means ‘absent’
FIG. 7 shows the effects of APPA, AP and PA on SA-β-Gal activity, CDKN1A gene expression levels, BECN-1 gene expression levels and BCL2L13 expression levels in primary human chondrocytes exposed to etoposide and OSM;
FIG. 7a: Effect of 20 μM Etoposide (Eto) plus 10 ng/ml Oncostatin M (OSM) on levels of the Senescent Associated Secretory Phenotype (SASP) marker β-Galactosidase;
FIG. 7b: Effects of APPA and its components on increased levels of the Senescent Associated Secretory Phenotype (SASP) marker β-Galactosidase induced by 20 μM Etoposide (Eto) plus 10 ng/ml Oncostatin M (OSM);
FIG. 7c: Effects of APPA and its components on increased gene expression of CDNK1A induced by 20 μM Etoposide (Eto) plus 10 ng/ml Oncostatin M (OSM);
FIG. 7d: Effects of APPA and its components on increased gene expression of BECN-1 induced by 20 μM Etoposide (Eto) plus 10 ng/ml Oncostatin M (OSM);
FIG. 7e: Effects of APPA and its components on increased gene expression of BCL2LK13 induced by 20 μM Etoposide (Eto) plus 10 ng/ml Oncostatin M (OSM);

### Drug Preparation

APPA was supplied in a prepared vial (2:7 ratio of AP:PA) while AP and PA were obtained from Sigma (Sigma-Aldrich, St Louis, MO, USA). All compounds were dissolved in Dimethyl Sulfoxide (DMSO) (Sigma-Aldrich) at final work concentration of 1 gr/ml and serial dilutions were developed.

### Chondrocyte Isolation

Written informed consent was obtained from all the subjects and approval was obtained from the local Ethics Committee of the Galician Health Administration (CEIC). All the procedures were conducted according to the principles expressed in the Helsinki Declaration of 1975, as revised in 2000.

Chondrocytes from human OA hip cartilage, were isolated as described in (Maneiro E, Martin M A, de Andres M C, López-Armada M J, Fernandez-Sueiro J L, del Hoyo P, et al. Mitochondrial respiratory activity is altered in osteoarthritic human articular chondrocytes. Arthritis and rheumatism. 2003; 48 (3): 700-8)) from 18 patients from total hip arthroplasty (5 male and 13 female patients with a mean±SD age of 86±7.17, 79.71±13.56 years respectively)

### T/C28a2 Chondrocytes

The immortalized human juvenile chondrocyte cell line T/C28a2 (25), was used in some experiments.

Human articular chondrocytes and T/C28a2 cell line, were equilibrated overnight in Dulbecco's Modified Eagle Medium (DMEM) (Gibco, Grand Island, NY, USA) supplemented with 10% fetal bovine serum (FBS), penicillin (100 U/ml) and streptomycin (100 μg/ml) (Gibco) in a humidified 5% CO₂ atmosphere at 37° C.

### Cell Culture Chondrocytes and T/C28a2 Cell Line:

Cells were plated at a density of 5×10⁴ cells/well in Corning® CellBIND® multiwell 96 plates (MW 96) (Corning, NY, USA) or 1.8×10⁵ cells/well in MW 12 plates (Corning, NY, USA) for analysis. Cells were left to equilibrate overnight in DMEM at 5% or 2% of FBS in 5% CO₂ at 37° C. Cells were stimulated with Etoposide at 20 UM for chondrocytes or 5 UM in the case of cell line (Sigma-Aldrich).

### EXAMPLES

### Example 1: Demonstration of Senomorphic Activity in Chondrocyte Cell Line TC28a2

The applicants found that in human chondrocyte cell line TC28a2, APPA had the capacity to decrease levels of the SASP marker β-Galactosidase—indicating a decrease in the number of senescent cells—but increase the total number of cells. Therefore, APPA showed a senomorphic effect on human chondrocyte cell line TC28a2.

A widely used marker of senescence is increased levels of senescence-associated β-galactosidase (SA-β-Gal) activity, shown by the quantification of fluorescein di-β-D-galactopyranoside (FDG) using flow cytometry.

The effect of APPA on number of senescent cells was therefore evaluated by the quantification of β-galactosidase activity using flow cytometry. β-galactosidase activity was detected using fluorescein di-β-D-galactopyranoside (FDG; Thermo Fisher, Waltham, MA, USA) by flow cytometry. Non-fluorescent FDG is sequentially hydrolysed by β-galactosidase, first to fluorescein monogalactoside and then to highly fluorescent fluorescein. Enzyme-mediated hydrolysis of FDG can be followed by the increase in fluorescence.

Cell cultures were pretreated with 5 μM Etoposide or 20 ng/ml interleukin 6 (IL-6) for 72 h (Sigma-Aldrich Merck KGaA, Darmstadt, Germany) to induce DNA damage as a genotoxic stress leading to cellular senescence and then with 10 nM bafilomycin A (Sigma-Aldrich) for 1 h to modulate intracellular pH. These conditions were evaluated with and without 10 μg/ml APPA for 24 h. Fluorescein di-β-D-galactopyranoside (10 UM) was then added to the pretreatment medium. At the end of incubation, cultures were washed with PBS, resuspended by trypsinization and analysed immediately using a FACScalibour flow cytometer (Becton Dickinson). Data were acquired and analysed with Cellquest software (Becton Dickinson). Each fluorescein signal was measured on an FL1 detector and β-galactosidase activity was estimated using the median fluorescence intensity (arbitrary units) of the cell population.

The median fluorescence of FDG (median±SEM) of human TC28a2 chondrocyte cells treated with APPA, and comparison to senescence controls (20 ng/ml IL-6 or 5 μM etoposide) is shown by FIG. 1 and Table 1. FIG. 1 demonstrates that APPA reduced the number of senescent cells, as indicated by the reduced SA-β-Gal activity, in both TC28a2 cells exposed to IL-6 and TC28a2 cells exposed to etoposide.

TABLE 1
Effect of APPA on levels of senescence-associated β-Galactosidase
(SA-β-Gal) in human TC28a2 chondrocyte cells;

| | | | P value | | |
|---|---|---|---|---|---|
| | | IL-6 + APPA 10 | (Mann | IL-6-72 h + APPA | P value (Mann |
| | IL-6 | μg/ml | Whitney) | 10 μg/ml-24 h | Whitney) |
| Mean ± | 0.9999 ± | 0.6896 ± 0.13 | * | 0.8014 ± 0.08 | * (p = 0.028) |
| SEM | 0.00004 | | (p = 0.028) | | |
| | | | P value | Eto-5 μM- | |
| | | Eto-5 μM + APPA | (Mann | 72 h + APPA-10 | P value (Mann |
| | Eto-5 μM | 10 μg/ml | Whitney) | μg/ml-24 h | Whitney) |
| Mean ± | 0.9999 ± | 0.6643 ± 0.11 | # | 1.411 ± 0.34 | ns (p = 0.34) |
| SEM | 0.00004 | | (p = 0.028) | | |

* = vs Basal;
# = vs Eto.

FIG. 2 demonstrates the effects of APPA, AP and PA on chondrocyte viability using DRAQ7.

A novel cell viability assay uses DRAQ7, a marker of apoptosis, necrosis and dead cells. DRAQ7 stains the nuclei in dead and permeabilised cells but not in intact live cells.

To determine the effects of APPA (10 μg/ml) AP (2.3 and 10 μg/ml) and PA (7.7 and 10 μg/ml) on chondrocyte viability, an assay based on DRAQ7™ (Thermo Fisher Scientific) was used. DRAQ7™ is a dye that can be used to show apoptosis, necrosis and dead cells, and the assay includes staining of nuclei in dead and permeabilised cells but not staining of intact live cells.

For the assay, cells were grown in flasks due to higher number of cells required. Following 24 h of different treatments, cells were trypsinised and centrifuged at 1500 rpm for 10 minutes (min). The harvested cells were suspended in 3 UM of the fluorescent dye DRAQ7™ (Thermo Fisher) and incubated in the dark, at room temperature (rt) for 10 min. The samples were run on CytoFLex flow cytometer (Beckman Coulter Inc. Ca, USA). At least 1×10⁴ cells per assay were measured. Data were analysed using CyExpert V 2.5 software (Beckman Coulter Inc). Positive control cells were treated with 5 μM Etoposide (Sigma-Aldrich) while negative control (Basal) cells were grown in fresh DMEM at 5% FBS. To establish the position of DRAQ7− and DRAQ7+ gates, one of each negative and positive control was stained with DRAQ7 dye and the other with PBS alone (data not shown).

FIG. 2 shows the effects of APPA (10 μg/ml), AP (2.3 and 10 μg/ml) and PA (7.7 and 10 μg/ml) on the percentage of (non-viable) cells positive for DRAQ7, used to estimate cell viability in the presence of APPA, AP or PA. The percentage of non-viable cells was never higher than 5.05%; this value was obtained in the 10 μg/ml AP condition. The DRAQ7 data shows that, at the concentrations used, APPA and its components AP and PA do not affect cell viability, thus excluding the possibility that the reduction in cell senescence is a result of cell toxicity. FIG. 3 shows the effect of APPA on the total viable cell number in chondrocyte cell line TC28a2.

After 8×10⁴ cells were cultured and had reached 70%-80% confluence, cells were pretreated with 5 μM Etoposide for 72 h, then the culture medium was changed for Dulbecco's Modified Eagle Medium (DMEM) at 2% foetal bovine serum (FBS) and cells were cultured in this condition for 48 h. Finally, APPA (10 μg/ml) was added for another 24 hours. After incubation, the cells were fixed in 4% paraformaldehyde (Sigma-Aldrich) for 10 min at room temperature, followed by incubation in 0.2% Tween20 (Sigma-Aldrich) for 5 min. This step was followed by 5 min incubation with the nuclear dye 2′-(4-ethoxyphenyl)-5-(4-methyl-1-piperazinyl)-2,5′-bi-1H-benzimidazole trihydrochloride (Hoechst 33258) (Sigma-Aldrich). After washing with phosphate-buffer (PB), cover slips were mounted on microscopy chamber slides using Prolong Gold Antifade Reagent Mountant (Thermo Fisher Scientific). Fluorescence was visualised and photographed under an Olympus BX61 fluorescence microscope. All samples were analysed in duplicate with 3-5 fields per well and mean values and standard deviations being calculated.

The results are shown in FIG. 3. When TC28a2 cells were incubated with 5 μM etoposide and 10 μg/ml APPA, the number of cells present in the culture increased in a statistically significant manner. APPA is therefore shown to produce its effect by a senomorphic mechanism of action; that is, to reduce levels of the SASP marker SA-β-Gal (indicating a decrease in the number of senescent cells) but increase the total number of viable cells. The susceptibility of cells to apoptosis was then analysed (FIG. 4). Cells were incubated in the presence of 2 μM 15 Etoposide or 20 ng/ml IL-6 for 72 h (Sigma-Aldrich) with or without 10 μg/ml APPA for 24 h. Cells were harvested by trypsinisation and resuspended in 1× annexin-binding buffer prior to adding 5 μL of annexin V-fluorescein isothiocyanate (FITC) and 5 μL of propidium iodide (PI; ImmunoStep, Salamanca, Spain). Following 15 min of incubation, cells were analysed with a FACsCalibur flow cytometer (Becton Dickinson, NJ, USA). Cells (1×10⁴) per assay were measured. Data were analysed using CellQuest software (Becton Dickinson). Apoptosis was analysed by counting cells stained simultaneously with annexin V-FITC and PI. This allowed the discrimination of intact cells (annexin V-FITC and PI negative) from cells in an early apoptotic state (annexin V-FITC positive and PI negative), late apoptotic state (annexin V-FITC and PI positive), and necrotic state (annexin V-FITC negative and PI positive). Results were expressed as a percentage of positive cells for each dye and represent the mean±standard error of mean (SEM) of three independent experiments.

FIG. 4 shows the effects of APPA on early (FIG. 4a) and late (FIG. 4b) apoptosis in cell line TC28a2 exposed to either 20 ng/ml IL-6 or 2 μM etoposide. APPA does not have any clear effect on either early or late apoptosis. Although an increase in apoptosis would have been an alternative route to reduce levels of SASP markers, this would have been associated with a decrease in the viable cell number. The fact that APPA increases the viable cell number, as shown by FIG. 3 above, aligns with the apoptosis data to show that the reduction in senescence does not occur as a result of an increase in apoptosis.

FIG. 5 and Table 2 show the effect of AP, PA and APPA on TC28a2 cell senescence induced by etoposide as determined by levels of SA-β-Gal. Levels of SA-β-Gal were evaluated via the quantification of fluorescein di-β-D-galactopyranoside (FDG) using flow cytometry, as in FIG. 1. When AP and PA were tested in parallel with APPA in TC28a2 cells exposed to etoposide, only APPA produced a significant reduction in SA-β-Gal activity, although PA 7.7 μg/ml approached significance (p=0.054). The effects produced by PA 10 μg/ml were not significant. APPA decreased the number of senescent TC28a2 cells (as indicated by a lower median fluorescence of FDG) more than its individual components AP or PA. The effect of APPA at 10 μg/ml was greater than expected based on (i) the effect of 10 μg/ml AP or 10 μg/ml PA and (ii) the additive effect of 2.3 μg/ml AP and 7.7 μg/ml PA. This is suggestive of a synergistic effect between AP and PA in the combined product.

TABLE 2
Effects of AP, PA and APPA on levels of senescence-associated β-Galactosidase
(SA-β-Gal) in human TC28a2 chondrocyte cells;

| | | | p value |
|---|---|---|---|
| | | Eto- | (Mann |
| | Basal | 5 μM | Whithney) |
| Mean ± | 1 | 1.144 ± | * |
| SEM | | 0.070 | (p = 0.107) |

| | | | | | | | Eto-5 | |
|---|---|---|---|---|---|---|---|---|
| | | Eto- | Eto - | Eto- | Eto-5 | p value | μM + | p value |
| | Eto-5 | 5 μM + AP | 5 μM + AP | 5 μM + PA | μM + PA | (Mann | APPA | (Mann |
| | μM | 10 μg/ml | 2.3 μg/ml | 10 μg/ml | 7.7 μg/ml | Whithney) | 10 μg/ml | Whithney) |
| Mean ± | 1.144 ± | 0.8948 ± | 0.9191 ± | 0.8902 ± | 0.8627 ± | ns | 0.6353 ± | # |
| SEM | 0.070 | 0.096 | 0.089 | 0.094 | 0.077 | (p = 0.054) | 0.109 | (p = 0.017) |

* = vs Basal;
# = vs Eto.

### Example 2—Demonstration of Senomorphic Activity in Primary Human Chondrocytes

To confirm the above data for chondrocyte cell line TC28a2, the applicants evaluated the senomorphic capacity of APPA using primary human chondrocytes.

To determine the optimal concentration of etoposide in human primary chondrocytes, concentrations of 2, 5 and 20 μM etoposide were combined with 10 ng/ml OSM and incubated for 48 hours. The results are shown in FIGS. 6a and 6b. The optimal etoposide concentration was evaluated as 20 μM.

The effects of APPA on the number of senescent cells as indicated by levels of SA-β-Gal are shown in FIG. 6c and Table 3. Levels of SA-β-Gal were evaluated via the quantification of fluorescein di-β-D-galactopyranoside (FDG) using flow cytometry, as in Example 1.

TABLE 3
Effects of APPA on number of senescent cells as determined
through levels of SA-β-Gal in primary human chondrocytes;

| | | Eto-20 | p value | Eto + | p value | APPA |
|---|---|---|---|---|---|---|
| | | μM + | (Mann | OSM + APPA | (Mann | 10 |
| | Basal | OSM | Whithney) | 10 μg/ml | Whithney) | μg/ml |
| Mean ± | 2.931 ± | 4.064 ± | * | 2.77 ± | # | 2.292 ± |
| SEM | 0.23 | 0.43 | (p = 0.037) | 0.13 | (p = 0.18) | 0.17 |

* = vs Basal;
# = vs Eto + OSM.
Data represented in the table corresponding to mean ± SEM, N = 8

CDKN1 (p21) is a genetic marker for cell senescence that codes for cyclin dependent kinase inhibitor 1A, a regulator of cell cycle progression. The expression of this gene is tightly controlled by the tumor suppressor protein p53. Activated p53 increases the expression of p21 which contributes to senescence. Studies in OA have suggested that cell cycle-related proteins play a role in its pathology. Cell-cycle arrest due to an increase in expression of genes that inhibit proliferation, the changes that occur in senescent cells can also result in the increased production of cytokines, growth factors, and matrix metalloproteinase (Loeser R F. Aging and osteoarthritis: the role of chondrocyte senescence and aging changes in the cartilage matrix. Osteoarthritis Cartilage. 2009 August; 17 (8): 971-9).

Evidence of cell senescence in tissues from older adults can be obtained by examining for the presence of senescence markers. These markers include histological staining for senescence-associated (SA) β-galactosidase (SA-β-Gal), SA heterochromatin, increased p53, p21, and p16 and reduced Wnt21 (Campisi J, d'Adda di Fagagna F. Cellular senescence: when bad things happen to good cells. Nat Rev Mol Cell Biol. 2007 September; 8 (9): 729-40. doi: 10.1038/nrm2233. PMID: 17667954).

The effects of APPA on expression levels of CDKN1 are shown in FIG. 6d and Table 4. Relative mRNA expression levels of CDKN1 were detected by quantitative real-time (RT)-PCR in presence of positive stimuli +/−10 μg/ml APPA. Data were obtained from eight independent donors. Values are presented as mean±SEM and analyzed by Mann Whitney test (*p≤0.05).

RNA extraction was achieved using TRIzol® (Sigma-Aldrich), following the manufacturer's protocol. 0.5 μg of RNA was reversely transcribed into cDNA using Super Script VILO (Thermofisher Scientific, Waltham, Massachusetts, USA) following the manufacturer's instructions. RT-PCR was developed in a LightCycler 480-II Instrument (Roche, Mannheim, Germany) using TaqMan Universal Master Mix (Roche). Analysis of the results was carried out using Qbase+ version 2.5 software (Biogazelle, Ghent, Belgium). Gene expression was calculated relative to the housekeeping gene Glyceraldehyde-3-Phosphate Dehydrogenase (GAPDH).

TABLE 4
Effects of APPA on expression levels of CDKN1 in primary human chondrocytes;

| | | Eto-20 | p value | Eto + OSM + | p value | APPA | p value |
|---|---|---|---|---|---|---|---|
| | | μM + | (Mann | APPA | (Mann | 10 | (Mann |
| | Basal | OSM | Whithney) | 10 μg/ml | Whithney) | μg/ml | Whithney) |
| Mean ± | 1.418 ± | 1.77 ± | ns | 0.9058 ± | # | 0.8843 ± | * |
| SEM | 0.11 | 0.17 | (p = 0.053) | 0.14 | (p = 0.012) | 0.17 | (p = 0.042) |
| | | | | | * | | |
| | | | | | (p = 0.042) | | |

* = vs Basal;
# = vs Eto + OSM.
Data represented in the table corresponding to mean ± SEM, N = 8

In primary human chondrocytes stimulated with 20 μM etoposide and 10 ng/ml oncostatin M, APPA significantly reduced levels of SA-β-Galactosidase, indicating a reduction in the number of senescent cells. APPA also reduced expression levels of CDKN1A (p21), a genetic marker for cell senescence. This is consistent with the TC28a2 results in Example 1.

FIG. 7 and Tables 5 and 6 show the effects of AP, PA and APPA on levels of SA-β-Gal and gene expression of CDKN1A (p21), BECN-1 and BCL2L13 in human chondrocytes.

The effects of AP, PA and APPA on the number of senescent cells as indicated by levels of SA-β-Gal are shown in FIG. 7b and Table 5. Levels of SA-β-Gal were evaluated via the quantification of fluorescein di-β-D-galactopyranoside (FDG) using flow cytometry, as in Example 1 and FIG. 6.

TABLE 5
Effects of APPA, AP and PA on number of senescent cells as indicated
by levels of SA-β-Gal in primary human chondrocytes;

| | | | p value |
|---|---|---|---|
| | | Eto-20 | (Mann |
| | Basal | μM + OSM | Whithney) |
| Mean ± | 1 ± | 1.542 ± | * |
| SEM | 2.7 e−07 | 0.244 | (p = 0.0286) |

| | Eto-20 | Eto + OSM + | p value | Eto + OSM + | Eto + OSM + | Eto + OSM + | Eto + OSM + |
|---|---|---|---|---|---|---|---|
| | μM + | APPA 10 | (Mann | AP-2.3 | AP-10 | PA-7.7 | PA-10 |
| | OSM | μg/ml | Whithney) | μg/ml | μg/ml | μg/ml | μg/ml |
| Mean ± | 1 ± | 0.8924 ± | # | 0.9947 ± | 1.01 ± | 0.9844 ± | 0.9622 ± |
| SEM | 2.79 e−07 | 0.043 | (p = 0.0286) | 0.056 | 0.029 | 0.046 | 0.043 |

* = vs Basal;
# = vs Eto + OSM.

When AP, PA and APPA were tested in human chondrocytes only APPA significantly reduced SA-β-Gal levels; AP and PA individually had no significant effects (see FIG. 7b). The effect of APPA at 10 μg/ml was greater than expected based on (i) the effect of 10 μg/ml AP or 10 μg/ml PA and (ii) the additive effect of 2.3 μg/ml AP and 7.7 μg/ml PA. This is again suggestive of synergism occurring between AP and PA in the combined product.

The effects of APPA, AP and PA on CDKN1A gene expression are shown in FIG. 7c and Table 6. Relative mRNA expression levels of cyclin dependent kinase inhibitor 1A (CDKN1) were detected by quantitative real-time (RT)-PCR in the same manner as above. Data were obtained from six independent donors. Values are presented as mean±SEM and analysed by Mann Whitney test (*p=0.05; #p≤0.05). * relative to basal condition #relative to 20 UM Etoposide+10 ng/ml oncostatin M.

TABLE 6
Effects of APPA, AP and PA on expression levels of CDKN1A
(p21) in primary human chondrocytes;

| | Mean ± SEM | P |
|---|---|---|
| Basal | 0.7738 ± 0.10 | |
| Eto 20 μM + OSM 10 ng/ml | 1.893 ± 0.48 | 0.0152* |
| Eto 20 μM + OSM 10 ng/ml + APPA 10 μg/ml | 0.9049 ± 0.07 | 0.026# |
| Eto 20 μM + OSM 10 ng/ml + AP 2.3 μg/ml | 1.0102 ± 0.25 | |
| Eto 20 μM + OSM 10 ng/ml + AP 10 μg/ml | 1.617 ± 0.32 | |
| Eto 20 μM + OSM 10 ng/ml + PA 7.7 μg/ml | 1.451 ± 0.18 | |
| Eto 20 μM + OSM 10 ng/ml + 10 μg/ml | 0.8635 ± 0.17 | |
| APPA 10 μg/ml | 0.9009 ± 0.24 | 0.0303# |

*= vs Basal;
#= vs Eto + OSM.

FIG. 7c and Table 6 show that APPA 10 μg/ml significantly reduced levels of CDKN1A (p21) expression (1.893±0.48 vs 0.904±0.07, p=0.05), as did PA 10 μg/ml (1.893±0.48 vs 0.869 ±0.17, p=0.05), whereas neither concentration of AP nor PA 7.7 μg/ml had any significant effect.

FIG. 7d shows the effects of APPA, AP and PA on gene expression of BECN-1. BECN-1 is a regulator of autophagy; the encoded protein, Beclin1, is a component of the phosphatidylinositol-3-kinase (PI3K) complex which mediates vesicle-trafficking processes. Autophagy is a self-degradative process that is important for balancing sources of energy at critical times during development and in response to cell stress. Although autophagy is not a type of cell death, it is important for cartilage homeostasis. Generally, autophagy promotes cell survival by adapting cells to stress conditions, but this process has also been considered as a non-apoptotic cell death program (Almonte-Becerril M, Navarro-Garcia F, Gonzalez-Robles A, Vega-Lopez M A, Lavalle C, Kouri J B. Cell death of chondrocytes is a combination between apoptosis and autophagy during the pathogenesis of Osteoarthritis within an experimental model. Apoptosis. 2010 May; 15 (5): 631-8. doi: 10.1007/s10495-010-0458-z. PMID: 20091349).

The reduction in BECN-1 expression by etoposide and OSM is significantly counteracted by APPA 10 μg/ml and PA 7.7 μg/ml, as shown in FIG. 7d, indicating an effect over autophagy. Interestingly, APPA alone reduces BECN-1 expression levels below those for non-stimulated chondrocytes (p=0.051); see FIG. 7d, column 8. FIG. 7e shows the effects of APPA, AP and PA on gene expression of BCL2L13. BCL2L13 encodes a mitochondrially-localized protein with conserved B-cell lymphoma 2 homology motifs. Overexpression of the encoded protein results in apoptosis. Apoptosis has been positively correlated with the severity of cartilage destruction and matrix depletion in human osteoarthritic tissue specimens (doi: 10.3390/ijms16036093). The anti-apoptotic proteins Bcl-2 and Bcl-XL inhibit cytochrome c (cyt-β) release. (Musumeci G, Castrogiovanni P, Trovato F M, Weinberg A M, Al-Wasiyah M K, Alqahtani M H, Mobasheri A. Biomarkers of Chondrocyte Apoptosis and Autophagy in Osteoarthritis. Int J Mol Sci. 2015 Aug. 31; 16 (9): 20560-75. doi: 10.3390/ijms160920560. PMID: 26334269; PMCID: PMC4613218.).

APPA does not seem to modulate the expression of BCL2L13. This aligns with the apoptosis results in FIG. 4, which indicate that APPA does not have any clear effect on either early or late apoptosis, and therefore that the reduction in senescence as a result of APPA administration does not occur via an increase in apoptosis.

### Example 3—Liquid Formulation

To produce a 1000 mg APPA formulation that is a stable liquid at room temperature, 777.8 mg paeonol (available from Sigma-Aldrich Gillingham) is mixed with 222.2 mg apocynin (available from Sigma-Aldrich Gillingham). The mixture is heated and 555.6 mg PEG 400 is added while the mixture is being agitated. A stable mixture is produced which does not solidify when stored at approximately 3° C. overnight.

### Example 4—Capsule Formulation—400 mg APPA Capsule

To produce a formulation containing approximately 400 mg of APPA, 311.1 mg paeonol (available from Sigma-Aldrich Gillingham) is mixed with 88.9 mg apocynin (available from Sigma-Aldrich Gillingham). The mixture is heated and 222.2 mg PEG 400 (available from Sigma-Aldrich Gillingham) is added while the mixture is being agitated. This yields a 0.533 ml liquid formulation of paeonol and apocynin which is stable at room temperature. This may be encapsulated within a soft gel capsule by methods known in the art to provide a pharmaceutical product in the form of a capsule.

### Example 5—Solid Formulation

To produce a solid formulation, the following preparation can be used to make type ‘O’ capsules (474.5 mg in total).

| EXAMPLE 5 | Unit mg/Capsules | Purpose |
|---|---|---|
| Paeonol (synthesised) | 311.1 mg | Active |
| Apocynin (synthesised) | 88.9 mg | Active |
| Excipient: Colloidal anhydrous | 0.5 mg | Lubricant |
| silica (silicon dioxide) | | |
| Excipient: Microcrystalline | 70 mg | Lubricant |
| cellulose | | |
| Excipient: Magnesium stearate | 4 mg | Lubricant |

There are hereinbefore disclosed compositions and uses as defined in the following numbered statements:

1. A composition comprising apocynin or an isomer thereof and paeonol or an isomer thereof for use in the treatment of senescence, wherein administration of the composition results in a decrease in the number of senescent cells and a simultaneous increase in the total viable cell number.

2. A composition comprising apocynin or an isomer thereof and paeonol or an isomer thereof for use in the treatment of senescence by a senomorphic mechanism of action.

3. A composition according to paragraph 1 or 2 wherein administration of the composition results in an increase in cell number.

4. A composition comprising apocynin or an isomer thereof and paeonol or an isomer thereof for use in the treatment of senescence by a senolytic mechanism of action.

5. A composition according to any preceding paragraph comprising apocynin and paeonol.

6. A composition according to any preceding paragraph wherein the ratio (by weight) of paeonol to apocynin is from 3:2 to 9:1.

7. A composition according to any preceding paragraph wherein the formulation is a liquid formulation.

8. A composition according to any of paragraphs 1-6 wherein the formulation is a solid formulation e.g. a pill, tablet or capsule.

9. A composition according to any preceding paragraph for use in the treatment of age-related diseases.

10. A composition according to any preceding paragraph for use in the treatment of osteoporosis.

11. A composition according to any preceding paragraph for use in the treatment of sarcopenia.

12. A composition according to any preceding paragraph for use in the treatment of chronic obstructive pulmonary disease.

13. A composition according to any preceding paragraph for use in the treatment of idiopathic pulmonary fibrosis.

14. A composition according to any preceding paragraph for use in the treatment of lung diseases such as pulmonary hypertension and pulmonary arterial hypertension.

15. A composition according to any preceding paragraph for use in the treatment of hepatic steatosis.

16. A composition according to any preceding paragraph for use in the treatment of kidney diseases such as acute kidney injury (AKI), chronic kidney disease (CKD), tubulointerstitial fibrosis (TIF), glomerulosclerosis and vascular sclerosis.

17. A composition according to any preceding paragraph for use in the treatment of atherosclerosis.

18. A composition according to any preceding paragraph for use in the treatment of type 1 diabetes.

19. A composition according to any preceding paragraph for use in the treatment of age-related macular degeneration.

20. A composition according to any preceding paragraph for use in the treatment of Alzheimer's disease, Parkinson's disease, frontotemporal dementia, amyotrophic lateral sclerosis and/or multiple sclerosis.

21. A composition according to any preceding paragraph wherein the composition is administered orally.

22. Use of apocynin or an isomer thereof and paeonol or an isomer thereof in the manufacture of a medicament for the treatment of senescence.

23. Use of apocynin or an isomer thereof and paeonol or an isomer thereof in the manufacture of a medicament for the treatment of age-related diseases.

24. Use of apocynin or an isomer thereof and paeonol or an isomer thereof in the manufacture of a medicament for the treatment of osteoporosis, sarcopenia, lung diseases such as chronic obstructive pulmonary disease (COPD), idiopathic pulmonary fibrosis (IPF), pulmonary hypertension (PH) and pulmonary arterial hypertension (PAH), liver diseases such as acute liver injury, chronic liver disease and hepatic steatosis, kidney diseases such as acute kidney injury (AKI), chronic kidney disease (CKD), tubulointerstitial fibrosis (TIF), glomerulosclerosis and vascular sclerosis, vascular diseases such as atherosclerosis, type 1 diabetes, age-related macular degeneration (AMD), Alzheimer's disease (AD), Parkinson's disease, frontotemporal dementia, amyotrophic lateral sclerosis or multiple sclerosis.

25. A method of treatment of age-related diseases, the method comprising a step of administering to a patient in need thereof a composition comprising apocynin or an isomer thereof and paeonol or an isomer thereof; or
a method of treatment of senescence, the method comprising a step of administering to a patient in need thereof a composition comprising apocynin or an isomer thereof and paeonol or an isomer thereof; or
a method of treatment of osteoporosis, sarcopenia, lung diseases such as chronic obstructive pulmonary disease (COPD), idiopathic pulmonary fibrosis (IPF), pulmonary hypertension (PH) and pulmonary arterial hypertension (PAH), liver diseases such as acute liver injury, chronic liver disease and hepatic steatosis, kidney diseases such as acute kidney injury (AKI), chronic kidney disease (CKD), tubulointerstitial fibrosis (TIF), glomerulosclerosis and vascular sclerosis, vascular diseases such as atherosclerosis, type 1 diabetes, age-related macular degeneration (AMD), Alzheimer's disease (AD), Parkinson's disease, frontotemporal dementia, amyotrophic lateral sclerosis or multiple sclerosis;
the method comprising a step of administering to a patient in need thereof a composition comprising apocynin or an isomer thereof and paeonol or an isomer thereof.

1. A composition comprising apocynin or an isomer thereof and paeonol or an isomer thereof for use in the treatment of senescence, wherein administration of the composition results in a decrease in the number of senescent cells and a simultaneous increase in the total viable cell number.

2. A composition comprising apocynin or an isomer thereof and paeonol or an isomer thereof for use in the treatment of senescence by a senomorphic mechanism of action.

3. A composition comprising apocynin or an isomer thereof and paeonol or an isomer thereof for use in the treatment of senescence by a senolytic mechanism of action.

4. A composition according to claim 1 comprising apocynin and paeonol.

5. A composition according to claim 1 wherein the ratio (by weight) of paeonol to apocynin is from 3:2 to 9:1.

6. A composition according to claim 1 wherein the formulation is a liquid formulation.

7. A composition according to claim 1 wherein the formulation is a solid formulation.

8. A composition according to claim 1 for use in the treatment of age-related diseases.

9. A composition according to claim 1 for use in the treatment of osteoporosis.

10. A composition according to claim 1 for use in the treatment of sarcopenia.

11. A composition according to claim 1 for use in the treatment of chronic obstructive pulmonary disease.

12. A composition according to claim 1 for use in the treatment of idiopathic pulmonary fibrosis.

13. A composition according to claim 1 for use in the treatment of lung diseases such as pulmonary hypertension and pulmonary arterial hypertension.

14. A composition according to claim 1 for use in the treatment of hepatic steatosis.

15. A composition according to claim 1 for use in the treatment of a kidney disease from the group consisting of acute kidney injury (AKI), chronic kidney disease (CKD), tubulointerstitial fibrosis (TIF), glomerulosclerosis and vascular sclerosis.

16. A composition according to claim 1 for use in the treatment of atherosclerosis.

17. A composition according to claim 1 for use in the treatment of type 1 diabetes.

18. A composition according to claim 1 for use in the treatment of age-related macular degeneration.

19. A composition according to claim 1 for use in the treatment of Alzheimer's disease, Parkinson's disease, frontotemporal dementia, amyotrophic lateral sclerosis and/or multiple sclerosis.

20. A composition according to claim 1 wherein the composition is administered orally.

21. Use of apocynin or an isomer thereof and paeonol or an isomer thereof in the manufacture of a medicament for the treatment of senescence.

22. Use of apocynin or an isomer thereof and paeonol or an isomer thereof in the manufacture of a medicament for the treatment of age-related diseases.

23. Use of apocynin or an isomer thereof and paeonol or an isomer thereof in the manufacture of a medicament for the treatment of osteoporosis, sarcopenia, lung diseases such as chronic obstructive pulmonary disease (COPD), idiopathic pulmonary fibrosis (IPF), pulmonary hypertension (PH) and pulmonary arterial hypertension (PAH), liver diseases such as acute liver injury, chronic liver disease and hepatic steatosis, kidney diseases such as acute kidney injury (AKI), chronic kidney disease (CKD), tubulointerstitial fibrosis (TIF), glomerulosclerosis and vascular sclerosis, vascular diseases such as atherosclerosis, type 1 diabetes, age-related macular degeneration (AMD), Alzheimer's disease (AD), Parkinson's disease, frontotemporal dementia, amyotrophic lateral sclerosis or multiple sclerosis.

24. A method of treatment of age-related diseases, the method comprising a step of administering to a patient in need thereof a composition comprising apocynin or an isomer thereof and paeonol or an isomer thereof.

25. A method of treatment of senescence, the method comprising a step of administering to a patient in need thereof a composition comprising apocynin or an isomer thereof and paeonol or an isomer thereof.

26. A method of treatment of osteoporosis, sarcopenia, lung diseases such as chronic obstructive pulmonary disease (COPD), idiopathic pulmonary fibrosis (IPF), pulmonary hypertension (PH) and pulmonary arterial hypertension (PAH), liver diseases such as acute liver injury, chronic liver disease and hepatic steatosis, kidney diseases such as acute kidney injury (AKI), chronic kidney disease (CKD), tubulointerstitial fibrosis (TIF), glomerulosclerosis and vascular sclerosis, vascular diseases such as atherosclerosis, type 1 diabetes, age-related macular degeneration (AMD), Alzheimer's disease (AD), Parkinson's disease, frontotemporal dementia, amyotrophic lateral sclerosis or multiple sclerosis, the method comprising a step of administering to a patient in need thereof a composition comprising apocynin or an isomer thereof and paeonol or an isomer thereof.
